(19) United States
(12) Patent Application Publication
(10) Pub. No.: US 2017/0106040 A1
(43) Pub. Date: Apr. 20, 2017

(54) COMPOSITION FOR PREVENTING AND TREATING MALE INFERTILITY, CONTAINING MIXED HERBAL EXTRACT AS ACTIVE INGREDIENT AND USE THEREOF

(71) Applicant: Industrial Cooperation Foundation Chonbuk National University, Jeollabuk-do (KR)

(72) Inventor: Jong Kwan PARK, Jeollabuk-do (KR)

(21) Appl. No.: 15/310,762

(22) PCT Filed: Apr. 8, 2015
(86) PCT No.: PCT/KR2015/003506

§ 371 Date: Nov. 12, 2016

(30) Foreign Application Priority Data
May 13, 2014 (KR) 10-2014-0057386

(51) Int. Cl. A61K 36/8962; A61K 36/39; A23L 33/00; A23L 33/10; A23P 10/40; A23P 10/28; A23P 10/30; A23L 2/52; A61K 36/746; A23L 33/105

(52) U.S. Cl. CPC A61K 36/8962; A61K 36/746; A61K 36/39; A23L 33/30; A23L 33/105; A23P 10/40; A23P 10/28; A23P 10/30; A23L 2/52; A23L 33/10; A61K 2236/333; A23V 2002/00

(57) ABSTRACT

A composition and health functional food for treating male infertility using a mixed herbal mixture. Through an in vitro sperm mobility promotion effect evaluation method experiment for investigating effects of an herbal extract of Morinda officinalis, Cuscuta seed, and/or Allium cepa of the present invention on changes in sperm count and mobility in human semen, it was verified that the composition increased sperm mobility. In addition, through an in vivo varicocele animal model evaluation method for investigating effects of the herbal extract on changes in sperm count and mobility in an in vivo varicocele animal model, it was verified that the composition samples of the present invention increased the sperm count in the semen obtained from spermiducts of a sample-administered group among varicocele-induced groups. Therefore, the herbal extract can be used in a pharmaceutical composition, health functional food, and health supplement food for treating male infertility.

### DETAILED DESCRIPTION OF THE INVENTION

### Technical Field

The present invention is related to a composition comprising the combined herb extract for treating and preventing male infertility, specifically, a composition comprising the combined herb extract of Morinda Officinalis root, Cuscuta seed, and Allium cepa bulb for the prevention and treatment of male infertility and the use thereof.

There has been reported that varicocele, the most treatable cause of male factor infertility, is characterized by the tortuosity and dilation of the veins of the pampiniform plexus within the spermatic cord and the prevalence of varicocele is 19% to 41% in men with primary infertility with male and more than 80% in men with secondary infertility (Agarwal A, Deppinder F, Cocuzza M, Agarwal R, Short R A, Sabanegh E, et al, (2007) Efficacy of varicocelectomy in improving semen parameters: new meta-analytical approach. Urology 70, pp 532-538; Agarwal A, Sharma R K, Desai N R, Prabakaran S, Tabanegh E. (2009) Role of Oxidative Stress in Pathogenesis of Varicocele and Infertility, Urology 73, pp 461-469).

Most frequently performed surgical procedure for the treatment of male infertility caused by varicocele, is varicocelectomy including non-microsurgical approach method, laparoscopic or microsurgical varicocelectomy, which have been performed from long years ago (Mehta A & Goldstein M. (2012) Microsurgical varicocelectomy: a review. Asian J. Androl., 15, pp 56-60). Although surgical correction of varicocele in infertile men has demonstrated improved parameters in 50-80% of patients performed by varicocelectomy (pregnancy rates, 31-74%), (Cheng D, Zheng X M, Li S W, Yang Z W & Hu L Q. (2006) Effects of epidermal growth factor on sperm content and motility of rats with surgically induced varicoceles. Asian J Androl 8, 713-717. Ding H, Tian J Q, Du W, Zhang L Y, Wang H Z & Wang Z P. (2012) Open non-microsurgical, laparoscopic or open microsurgical varicocelectomy for male infertility: a meta-analysis of randomized controlled trials. BJU Int 110, 1536-1542; Fisch H & Hyun G. (2012) Varicocele repair for low testosterone. Curr Opin Urol 22, 495-498; Fretz P C & Sandlow J I. (2002) Varicocele: current concepts in pathophysiology, diagnosis, and treatment. Urol Clin North Am 29, 921-937; Mehta A & Goldstein M. (2013) Microsurgical varicocelectomy: a review. Asian J Androl 15, 56-60; Tanriverdi O, Miroglu C, Horasanli K, Altay B, Caliskan K C & Gumus E. (2006) Testicular blood flow measurements and mean resistive index values after microsurgical and high ligation varicocelectomy. Urology 67, 1262-1265), some reports have issued that varicocele repair does not seem to be an effective treatment for male or unexplained subfertility such as increased pregnancy failure rate of fertile postoperation etc (Evers J L & Collins J A. (2003) Assessment of efficacy of varicocele repair for male subfertility: a systematic review. The Lancet 361, 1849-1852; Ficarra V, Cerruto M A, Liguori G, Mazzoni G, Minucci S, Tracia A, et al. (2006) Treatment of varicocele in subfertile men: the Cochrane review a contrary opinion. Eur Urol 49, 258-263).

Although the exact mechanism by which the impaired testicular function in patients with varicocele causes to infertility, has not fully elucidated yet till now, some of hypothetical mechanisms have been suggested to study various varicocele related testicular dysfunctions including blood flow alteration in the testis, scrotal temperature elevations, abnormal hormone values, excessive reactive oxygen species (ROS) and so on (Agarwal A, Sharma R K, Desai N R, Prabakaran S, Tavares A & Sabanegh E. (2009) Role of Oxidative Stress in Pathogenesis of Varicocele and Infertility. Urology 73, 461-469; Fretz P C & Sandlow J I. (2002) Varicocele: current concepts in pathophysiology, diagnosis, and treatment. Urol Clin North Am 29, 921-937).

ROS (Reactive oxygen species) acting as a modulator of vital physiological intracellular processes is one of by-product reproduced from oxygen metabolism and energy production process. Small amount of ROS plays important role in controlling the spermatozoa function in male reproductive tract. Under abnormal condition, the oxidative stress follows where the increase of ROS level or reduction of anti-oxidative ability occurs. Specifically, oxidative stress causes to the modified level of expressed protein in the sperm, resulting in the deficiency of molecule and gene (Sharma R, Agarwal A, Mohanty G, Hamada A J, Gopalan B, Willard B, et al. (2013) Proteomic analysis of human spermatozoa proteins with oxidative stress. Reprod Biol Endocrinol 11, 48).

Normally, the pregnancy rate of healthy couple reaches to 20-25% at the 1^{st} month, 75% at the half year, and 85-90% at one year after the sexual relation. In clinical, the ratio of infertile patients among the married couples is presumed to become about 13-20% in the married couples (about 1,400,000 couples) in South Korea under the condition that the “infertility” is defined by the failed cases to pregnancy one year after the sexual relation. Among them, 35-50% infertility is caused by the health problem of men and about 30% of the problem results from the reduced motility of spermatozoa. Male infertility is one of the important and severe diseases of the outpatients, which becomes to 4% of the number of outpatients in urological hospital.

Recently, various ART (assisted reproductive technique) such as IVF (in vitro fertilization), micromanipulation technique etc have been developed due to the advanced reproductive biology or ART. However, the techniques have also some disadvantages and problems, for example, low pregnancy rate of only 20 to 40%, need for excessive expense, difficulty in applying on woman because of pain complaint, occurrence of complication and so on.

External fertilization also has the problem such as gene contamination, excessive expense etc.

Accordingly, the most safe and ideal target to increase the pregnancy rate of married couple, has been focused to develop the effective therapy for increasing the spontaneous pregnancy rate in the field of urological area and gynecological area till now.

In spite of the developed therapy of infertility, the effective treating agent to treat a male infertility and improving the sperm motility has not yet developed till now and there has been few studies on the drug which can improve the pregnancy rate. One of the current approaches to improve the semen parameters of men with low sperm motility, L-carnitine has been developed as a treating agent however the drug have been reported to show little apparent efficacy (Mark Sigman, Stacy Glass, Janice Campagnone, John L. Reyor, Carnitine for the treatment of idiopathic asthenospermia: a randomized, double-blind, placebo-controlled trial, Fertility and Sterility Vol 85, No 5, 2006)

Morinda Officinalis root, a root of Morinda Officinalis belongs to Rubiaceae family and distributed at Southern region of China has been reported to comprise vitamin C, saccharide etc (B. S. Chung et al., YoungRim Press, 2^{nd} Edition, Dohae Hyang Yak Dae Sa Jeon., pp 927-928, 1998).

Cuscuta seed, a seed of Cuscuta japonica, or the like belongs to Convolvulaceae family has been reported to comprise resin glycoside, saccharide, vitamin A, beta-carotene, gamma-carotene, teraxanthin, lutein etc (B. S. Chung et al., YoungRim Press, 2^{nd} Edition, Dohae Hyang Yak Dae SaJeon., pp 882-883, 1998).

Allium cepa bulb, a bulb of Allium cepa belongs to Liliaceae family which is originated in Siberia or Altai has been reported to comprise thiol, dimethyl disulfide, diallyl disulfide, caffeic acid, ferulic acid, sinapic acid etc (B. S. Chung et al., YoungRim Press, 2^{nd} Edition, Dohae Hyang Yak Dae Sa Jeon., pp 155-156, 1998).

However, there has been not reported or disclosed about the therapeutic effect of the extract of combined herbs consisting Morinda officinalis root, Cuscuta seed and Allium cepa bulb as described above on male infertility disease in any of above cited literatures, the disclosures of which are incorporated herein by reference.

Therefore, the present inventors have endeavored to find the effective herb formulation for treating and preventing male infertility disease and the combined herb composition shows potent treating effect on human infertility, which is confirmed by various experiments, for example, In vitro sperm quality promoting effect evaluation as well as in vivo varicocele animal model evaluation method for investigating effects of the herbal extract on changes in sperm count and mobility in an in vivo varicocele animal model. Finally, the present inventors have found that the above-described combined herb extract is effective in treating and preventing male infertility disease.

### Technical Solution

The technical solution to solve the problem of the background art is for the development of novel approach to increase the pregnancy rate through the promotion of sperm motility and then to recover the previous high pregnancy rate in the end.

According to one aspect, the present invention provides a pharmaceutical composition comprising the combined herb extract of Morinda Officinalis root, Cuscuta seed, and Allium cepa bulb, as an active ingredient for preventing and treating a male infertility disease.

The present invention also provides a health functional food comprising the combined herb extract of Morinda Officinalis root, Cuscuta seed, and Allium cepa bulb for the prevention or improvement of male infertility disease as an active ingredient to prevent and improve a male infertility disease.

The extract disclosed herein comprises the combined herb extract, i.e., Morinda Officinalis root, Cuscuta seed, and Allium cepa bulb with the mixed ratio based on the dried weight of each herb (w/w) ranging from 0.01-100:0.01-1:1-100(w/w), preferably, 0.1-50:0.1-1:1-50(w/w), more preferably, 1-30:1:1-30 (w/w) in the present invention.

Specifically, the term “extract” disclosed herein comprises the extract soluble in distilled water, C₁-C₄ alcohols or the mixtures thereof, preferably, water, ethanol or the mixture thereof.

Specifically, the term “male infertility disease” disclosed herein comprises all the male infertility disease, for example, not intended to limit thereto, a male infertility disease caused by the adverse response of anti-cancer agent such as cisplatin, vinblastine, bleomycin and the like; azoospermia (sperm count: none), oligospermia (sperm count: <2×10⁷/ml), asthenospermia (ratio of high motile sperm: <40%), teratospermia (ratio of normal sperm: <40%) and the like (See, Korean Society for sexual medicine and andrology, Textbook of andrology, 2010).

An inventive herb composition may be prepared in accordance with the following preferred embodiment.

For example, the present invention also provides a method for preparing the inventive herb extract comprising the steps of; washing dried Morinda Officinalis root, Cuscuta seed, or Allium cepa bulb, respectively and adding 1 to 20-fold, preferably, 1 to 10-fold volume of distilled water, alcohols such as methanol, ethanol, and the like, or the mixtures thereof, preferably, distilled water, ethanol or the mixture thereof, thereto at 1^{st} step; extracting each solution with the extraction method by the extraction with hot water, cold water, or ultra-sonication extraction, preferably, hot water extraction at the temperature ranging from 50° C.-120° C., preferably, about 80° C.-100° C., for the period ranging from 1 to 5 hours, preferably, 2 to 4 hours at 2^{nd} step; repeating the above-described extraction process to collect each filtrate with filtration, drying through freeze drying, natural air drying or hot air drying process, preferably freeze drying process to obtain respective dried extract of Morinda Officinalis root, Cuscuta seed, or Allium cepa bulb at 3^{rd} step; and mixing the respective dried extract of each herb(Morinda Officinalis root:Cuscuta seed:Allium cepa bulb) with the mixed ratio based on the dried weight of each herb (w/w) ranging from 0.01-100: 0.01-1:1-100(w/w), preferably, 0.1-50:0.1-1:1-50(w/w), more preferably, 1-30:1:1-30 (w/w) to prepare inventive extract of the present invention.

It is another object of the present invention to provide a process for preparing the extract of the present invention as described above for the preparation of composition effective in treating or preventing the purposed diseases.

It is still another object of the present invention to provide a pharmaceutical composition or health functional food comprising the herb extract of the above-mentioned herb obtained by the above described process as an active ingredient for preventing and treating male infertility disease.

The inventive composition of the present invention shows potent treating effect on human infertility, which is confirmed by various experiments, for example, In vitro sperm quality promoting effect evaluation as well as in vivo varicocele animal model evaluation method for investigating effects of the herbal extract on changes in sperm count and mobility in an in vivo varicocele animal model, it was verified that the inventive combined extract showed more potent increasing effect on the sperm count and sperm motility. Therefore, the herbal extract of the present invention can be usefully used in a pharmaceutical composition, health functional food, and health supplement food for preventing and treating male infertility.

The pharmaceutical composition for treating purposed diseases could contain about 0.01 to 99 w/w % the above herb extract of the present invention based on the total weight of the composition.

However, the amount and each component of the above-mentioned composition can be varied with the patient's condition, development of patient's disease, the sort of disease etc.

The inventive composition may additionally comprise conventional carrier, adjuvants or diluents in accordance with a using method.

The herb composition according to the present invention can be formulated in oral dosage form such as powder, granule, tablet, capsule, suspension, emulsion, syrup, aerosol and the like; topical preparation; or injection solution. The herb composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, microcrystalline cellulose, polyvinyl pyrrolidone, water, methylhydroxy benzoate, propylhydroxy benzoate, magnesium stearate and mineral oil. The formulations may additionally include excipients such as fillers, bulking agents, binders, wetting agents, disintegrating agents, surfactants, diluents and the like. The solid oral dosage form comprises tablet, pill, powder, granule, capsule and the like and the solid oral dosage form is prepared by adding at least one excipient such as starch, calcium carbonate, sucrose, lactose or gelatin and the like to the herb extract. Lubricant such as magnesium stearate or talc may be used. The aqueous oral dosage form comprises suspension, oral solution, emulsion, syrup and the aqueous oral dosage form may comprise several excipients such as wetting agents, sweetener flavoring agents, preservatives, as well as water, liquid paraffin. The parenteral dosage form comprises sterilized aqueous solution, non-aqueous solvent, suspension, emulsion, lyophilized preparation, suppository, and the like. Suitable examples of the carriers include propylene glycol, polyethylene glycol, vegetable oils such as olive oil, injectable ester such as ethyl oleate. Base for suppository may include witepsol, macrogol, tween 61, cacao butter, laurin, glycerogelatine etc., but are not limited to them.

The desirable dose of the inventive composition varies depending on the condition and the weight of the subject, severity, drug form, route and period of administration, and may be chosen by those skilled in the art. However, in order to obtain desirable effects, it is generally recommended to administer at the amount ranging 0.01 mg/kg to 10 g/kg, preferably, 1 mg/kg to 1 g/kg by weight/day of the inventive composition of the present invention. The dose may be administered in a single or multiple doses per day.

The pharmaceutical composition of present invention can be administered to a subject animal such as mammals (rat, mouse, domestic animals or human) via various routes. All modes of administration are contemplated, for example, administration can be made orally, rectally or by intravenous injection.

It is still another object of the present invention to provide a sperm motility improving agent comprising the combined herb extract of Morinda Officinalis root, Cuscuta seed, and Allium cepa bulb, as an active ingredient for preventing and treating male infertility disease.

It is still the other object of the present invention to provide a treating agent for male infertility by way of improving sperm motility comprising the combined herb extract of Morinda Officinalis root, Cuscuta seed, and Allium cepa bulb as an active ingredient.

It is still the other object of the present invention to provide a sperm storing additive for IVF (in vitro fertilization) comprising the combined herb extract of Morinda Officinalis root, Cuscuta seed, and Allium cepa bulb for improving fertilization rate of IVF.

It is the other object of the present invention to provide a method of treatment comprising administering of the composition comprising the combined herb extract of Morinda Officinalis root, Cuscuta seed, and Allium cepa bulb to a subject in need of treatment or prevention of male infertility disease.

It is another object of the present invention to provide a use of the combined herb extract of Morinda Officinalis root, Cuscuta seed and Allium cepa bulb as an active ingredient for manufacture of medicament employed for treating or preventing male infertility disease in human or mammal.

In accordance with one aspect of the present invention, there provided a health functional food comprising the combined herb extract of Morinda Officinalis root, Cuscuta seed, and Allium cepa bulb for the prevention or improvement of male infertility disease as an active ingredient.

The term “a health functional food” defined herein” comprises the functional food having enhanced functionality such as physical functionality or physiological functionality by adding the extract of the present invention to conventional food to prevent or improve the purposed diseases in human or mammal and stipulated by the Law for Health Functional Foods 6727 in Republic of Korea.

The health functional food composition for preventing and improving purposed diseases could contain about 0.01 to 95 w/w %, preferably 1 to 80 w/w % of the above herb composition of present invention based on the total weight of the composition.

Moreover, the inventive extract of the present invention also can be used as a main component or additive and aiding agent in the preparation of various functional health food and health supplement food for the prevention or improvement of male infertility disease or the improvement of testicular function.

The inventive health functional food may be prepared and processed by the form of pharmaceutically acceptable dosage form such as powder, granule, tablet, capsule, pills, suspension, emulsion, syrup and the like; or the functional health food form such as tea bag, leached tea, health beverage type and the like.

It is the other object of the present invention to provide a health supplement food comprising the combined herb extract of Morinda Officinalis root, Cuscuta seed, and Allium cepa bulb, as a main component, for the prevention or improvement of male infertility diseases.

The above-mentioned term “as a main component” means that the above health supplement food comprises about 30 to 99 (w/w %), preferably 50 to 99 (w/w %), more preferably 70 to 99 (w/w %) of the inventive extract of present invention based on the total weight of the composition.

When the combined herb extract of the present invention is used as a component in the health functional beverage composition, the health functional beverage composition can comprise other component such as flavoring agent or natural carbohydrate without limits like that typical beverage composition. Examples of the natural carbohydrate comprise monosaccharide such as glucose, fructose etc; disaccharide such as maltose, sucrose etc; and polysaccharide, for example, sugar such as dextrin, cyclodextrin, and sugar alcohol such as xylitol, sorbitol, erythritol. Natural flavoring agent (thaumatin, stevia extract (rebaudioside A, glycyrrhizin, etc)) and synthetic flavoring agent (saccharin, aspartame, etc) may be added in the health functional beverage composition. The amount of natural carbohydrate generally ranges from about 1 to 20 g, preferably about 5 to 12 g per 100 ml of the present composition.

When the combined herb extract of the present invention is used as a food additive of the health food, the combined herb extract may be added intact or used with other food ingredient according to general process. Examples of the food comprises meat products, sausage, bread, chocolate, candy, snack, cracker, biscuit, pizza, ramen, noodle products, chewing gum, dairy products such as ice cream, soup, beverage, tea, drinks, alcohol drink, vitamin complex etc, but not intended herein to limit thereto, for preventing or improving of purposed disease.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Also, above described extract can be added to food or beverage for prevention and improvement of purposed disorder. The amount of above described extract in food or beverage as a functional health food or health supplement food may generally range from about 0.01 to 15 w/w % of total weight of food for functional health food composition. And the extract of the present invention may be added 0.02 to 5 g, preferably 0.3 to 1 g per 100 ml in health beverage composition.

### Advantageous Effects

As described in the present invention, inventive combined herb composition shows potent treating effect on human infertility, which is confirmed by various experiments, for example, In vitro sperm quality promoting effect evaluation as well as in vivo varicocele animal model evaluation method for investigating effects of the herbal extract on changes in sperm count and mobility in an in vivo varicocele animal model, it was verified that the inventive combined extract showed more potent increasing effect on the sperm count and sperm motility. Therefore, the herbal extract of the present invention can be usefully used in a pharmaceutical composition, health functional food, and health supplement food for preventing and treating male infertility.

### DESCRIPTION OF DRAWINGS

The above and other objects, features and other advantages of the present invention will more clearly understood from the following detailed description taken in conjunction with the accompanying drawings, in which;

FIG. 1 shows the improving effect of test sample on the human seminal count and human seminal motility in human semen (the preliminary test on each test samples of CINTM1 (10:1:10), CINTM1 (5:1:5), and CINTM1 (1:1:1) was performed before the test);

FIG. 2 shows the improving effect of test sample on the human seminal count and human seminal motility in human semen between extract of each herb and combined herb (CINTher-1: extract of Morinda Officinalis root; CINTher-2: extract of Cuscuta seed; CINTher-3: extract of Allium cepa bulb; CINTM1: extract of combined herb mixed with 10:1:10, respectively);

FIG. 3 shows the improving effect of test sample on the sperm count in the varicocoele-induced animal group (CTR: Control, OP: Operation);

FIG. 4 presents the improving effect of test sample on the sperm motility in the varicocoele-induced animal group (CTR: Control, OP: Operation).

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Examples and Experimental Examples are intended to further illustrate the present invention without limiting its scope.

### Example 1

### Preparation of Each Component

1-1. The Extract of Morinda officinalis Root

3,000 g of dried root of Morinda officinalis purchased from Kyung-dong market (Dukhyundang Pharmacy) located in Seoul were washed and added to 6 L of 95% ethanol to perform extraction with sonication extraction for 90 mins 3 times. The residue was filtered with filter paper to afford extract and the filtrated extract was concentrated under vaccuo. The concentrated extract was dried with freeze dryer (ScanVac, Labogene) to afford 139.4 g (Yield: 4.6%) of dried extract of Morinda officinalis root (designated as CINTher-1 hereinafter), which is used as a comparative test sample by dissolving into physiological saline solution and vigorously stirring for 2 mins to store at 4° C. prior to use in following experiment.

### 1-2. The Extract of Cuscuta Seed

5.2 kg of dried seed of Cuscuta Chinensis purchased from Kyung-dong market (Backjangsaeng Pharmacy) located in Seoul were washed, pulverized, and added to 5 L of 95% ethanol to perform extraction with sonication extraction for 90 mins 2 times. The residue was filtered with filter paper to afford extract and the filtrated extract was concentrated under vaccuo. The concentrated extract was dried with freeze dryer (ScanVac, Labogene) to afford 264.3 g (Yield: 4.36%) of dried extract of Cuscuta seed (designated as CINTher-2 hereinafter), which is used as a comparative test sample by dissolving into physiological saline solution and vigorously stirring for 2 mins to store at 4° C. prior to use in following experiment.

1-3. The Extract of Allium cepa Bulb

1,000 g of bulb of Allium cepa purchased from Changnyeong market located in Kyungsangnamdo (Korea) were washed, dried and added to 5 L of 70% ethanol to perform extraction with reflux extraction for 2 hours at 70° C. The residue was filtered with filter paper to afford extract and the filtrated extract was concentrated under vaccuo. The concentrated extract was dried with freeze dryer (ScanVac, Labogene) to afford 13.8 g (Yield: 1.38%) of dried extract of Allium cepa bulb (designated as CINTher-3 hereinafter), which is used as a comparative test sample by dissolving into physiological saline solution and vigorously stirring for 2 mins to store at 4° C. prior to use in following experiment.

### Example 2

### Preparation of Combined Formulation (CINTM1)

The dried extract of Morinda officinalis root, Cuscuta seed and Allium cepa bulb prepared in Example 1 was thoroughly mixed with different mixed weigh ratios (10:1:10/5:1:5/1:1:1) using by mixer (Vortex genie-2, scientific industries, USA) to obtain the various kinds of invention formulation (designated as “CINTM1” hereinafter), which are used as a test samples in following experiment.

### Experimental Example 1

### In Vitro Sperm Quality Promoting Effect Evaluation

In order to investigate the effect of the inventive extract obtained in Examples on the sperm number and sperm motility of human spermatozoa, following in vitro experiment was performed according the procedure disclosed in the literature (Martin Blomberg Jensen, Poul J. Bjerrum, Torben E. Jessen, John E. Nielsen, Ulla N. Joensen, et al. (2011) Vitamin D is positively associated with sperm motility and increases intracellular calcium in human spermatozoa. Human Reproduction, Vol. 26, No. 6, pp. 1307-1317).

### 1-1. Procedure

After determining the number of sperm and sperm motility of human semen delivered from called patients volunteers suffering from infertility (Urology division of Chonbuk National University Hospital in South Korea, 20 to 50 years old, 85 patients), the semen was poured into incubator maintaining 37° C. The liquefaction of the semen was sufficiently completed and then 200 μL of test sample was added to 20 μL of semen to the extent to reach 2 mg/ml of final concentration. Three hour after the addition, the sperm motility was determined by using counting chamber (MAKLER counting chamber, self-medical instruments, Haifa, Israel) and microscope (Axiovert25, ZEISS, USA).

3H buffer solution {Hams F-10(N6635, Sigma chemical Co/USA)+0.4% HAS (A1635, Sigma Chemical Co. USA)+12 mM HEPES (H4034, Sigma Chemical Co. USA) [3H, pH 7.4]} was used as a control group. The test samples were also dissolved in the same buffer solution as mentioned the above, adjusted to pH 7.4 and centrifuged by centrifuge apparatus (A32010(1), LABOGENE, 13,000 rpm, 2 mins, 4° C.) to afford the supernatant for determination of sperm count and motility.

The sperm motility was calculated according to following empirical formulae 1.

Percentage of motility=A (the mean number of motile sperm)/B (total number of sperms) [Empirical formulae 1]

### 1-2. Test Result

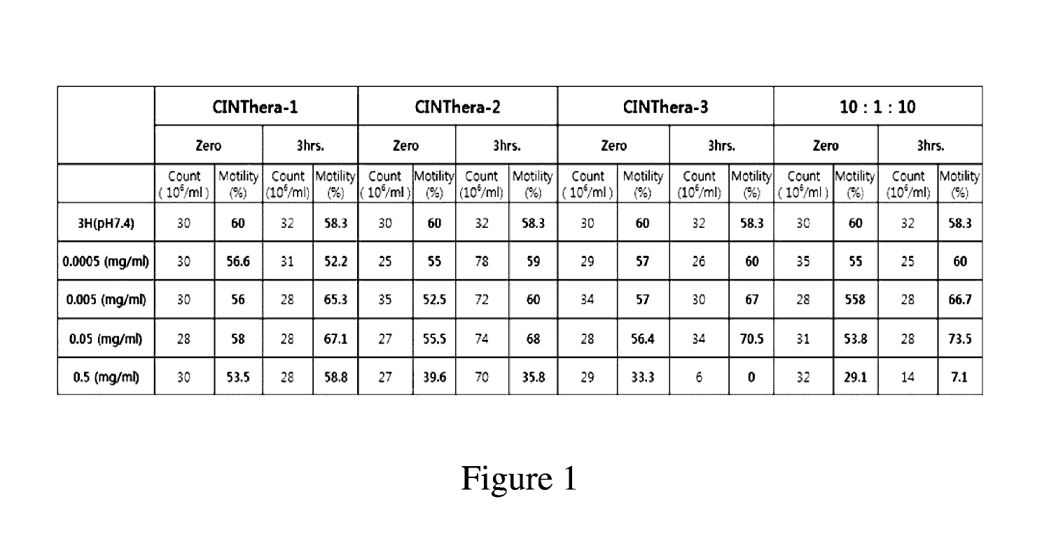

The sperm motility in the group treated with three kinds of inventive combinations, i.e., CINTM1(10:1:10), CINTM1(5:1:5), and CINTM1(1:1:1) was sharply increased by 70% in the group CINTM1(10:1:10) at the concentration of 0.05 mg/ml, 62% in the group CINTM1(5:1:5) at the concentration of 0.5 mg/ml and 63% in the group CINTM1(1:1:1) at the concentration of 0.5 mg/ml, respectively. Among the test sample groups, the CINTM1 (10:1:10) group showed most potent increasing effect on sperm motility comparing with the other groups with different mixed ratio and the comparative test group treated with the extract of sole herb, i.e., CINTher-1, CINTher-2 and CINTher-3 (See FIG. 1).

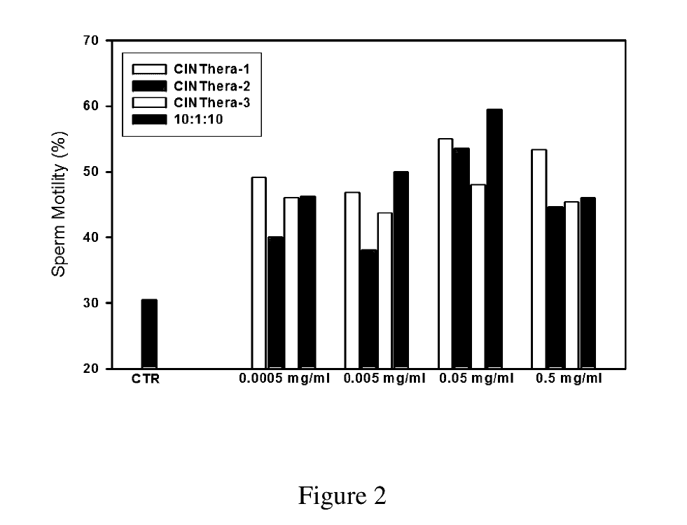

Specifically, the test groups treated with CINTM1(10:1:10) showed unexpectedly superior effect on the sperm motility at the same concentration (59.5%, 0.05 mg/ml) comparing with the comparative test group treated with the extract of sole herb, i.e., CINTher-1(55%), CINTher-2 (53.5%) and CINTher-3(48.0%), respectively. (See FIG. 2).

Accordingly, it has been confirmed that the combination of each herb (CINTM1) showed synergistic effect on sperm motility activity.

### Experimental Example 2

### In Vivo Sperm Quality Promoting Effect Evaluation

In order to investigate the effect of the inventive extract obtained in Examples on the sperm number and sperm motility of human spermatozoa, following in vivo experiment was performed according the procedure disclosed in the literature (Zhang L T, Kim H K, Choi B R, Zhao C, Lee S W, Jang K Y, et al. (2014) Analysis of testicular-internal spermatic vein variation and the recreation of varicocoele in a Sprague-Dawley rat model. Andrology. 2, 466-73).

### 2-1. Establishment of In Vivo Varicocoele Animal Model

In order to establish In vivo varicocoele animal model for use in following experiment, 9 weeks old Sprague-Dawley rats weighing 300 to 350 g (purchased from KOATECH Co. Ltd located at Pyeongtaek city, KOREA) were bred after acclimating to environment for 1 week. The rats divided with four rats per cage, were fed standard rat chow and freely access to purified water. The rats were maintained in the animal facility under constantly controlled environmental conditions (internal temperature: 18° C. to 23° C., relative humidity: 40 to 60%) and 12 hours light-dark cycle).

Each rat was intraperitoneally anaesthetized using by a mixture of Ketamine (50 mg/kg) and Xylazine (25 mg/kg) and the renal vein connected to venae cava was exposed after approaching left renal through a midline abdominal incision. The left renal vein was ligated with a 5.0 nylon suture with a metal probe (outer diameter: 0.85 mm) and the metal probe was withdrawn, which leads to 50% reduction in the diameter of the left renal vein and a consistent constriction of the vessel at the point of the ligation. ISV (Internal spermatic vein) branch was ligated to induce varicocoele for use as a varicocoele-induced animal group and four weeks after the ligation, the test samples dissolved in physiological saline solution were orally administrated into the experimental rats.

### 2-2. Evaluation of Potency in in Vivo Animal Model

2 ml/day of test samples or physiological saline solution (PSA) had been orally administrated into two test groups for 28 days after the operation, i.e., varicocoele-induced animal group {(1) PSA: 2 ml/day, (2) CINTM1(10:1:10):100 mg/kg/2 ml/day, (3) CINTM1(10:1:10):200 mg/kg/2 ml/day} and normal control group {(1) PSA:2 ml/day, (2) CINTM1(10:1:10):100 mg/kg/2 ml/day, (3) CINTM1(10:1:10):200 mg/kg/2 ml/day} and then the weight of rats was weighed. The seminal duct of rat was delivered under general anesthesia. The sperm count and motility in the seminal duct were determined and the weight of bilateral testis and epididymis was weighed to compare.

### 2-3. Test Result

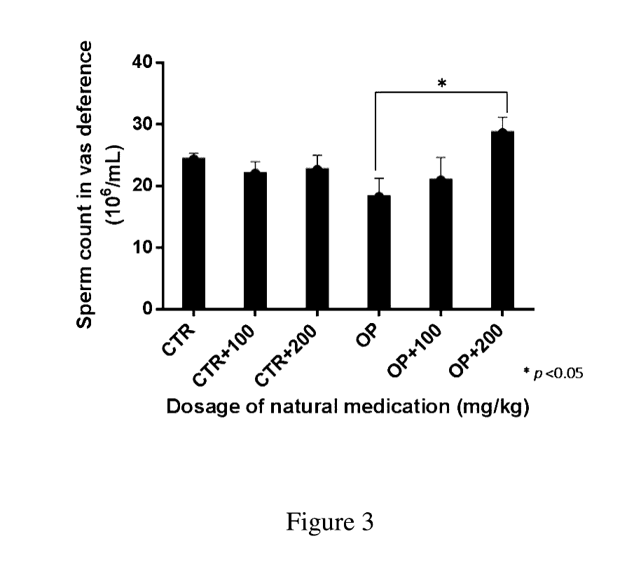

As shown in FIG. 3, the sperm count in the varicocoele-induced animal group treated with inventive combination showed sharply increased comparing with the group treated with PSA. The sperm count in the varicocoele-induced animal group treated with inventive combination was 20.91±12.25×10⁶/ml (100 mg/kg/2 ml/day) and 28.63±8.90×10⁶/ml (200 mg/kg/2 ml/day) while that in the varicocoele-induced animal group treated with PSA was only 18.25±9.53×10⁶/ml. (See FIG. 3).

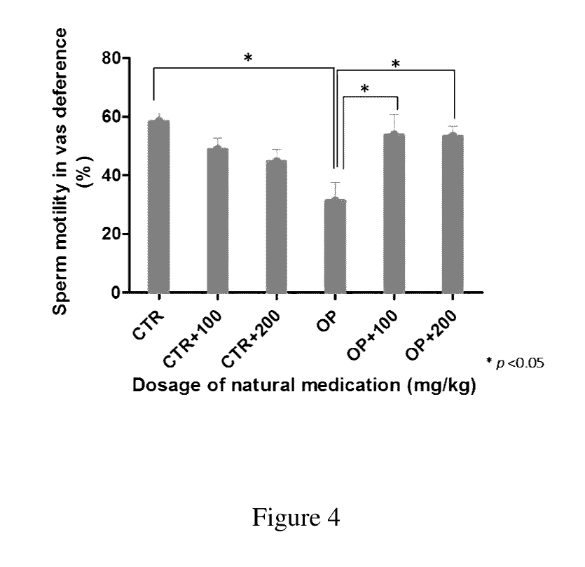

As shown in FIG. 4, the sperm motility in the varicocoele-induced animal group treated with inventive combination showed sharply increased comparing with the group treated with PSA. The sperm motility in the varicocoele-induced animal group treated with inventive combination was 53.8±20.7% (100 mg/kg/2 ml/day) and 53.3±12.1% (200 mg/kg/2 ml/day) while that in the varicocoele-induced animal group treated with PSA was only 31.3±18.8%. (See FIG. 4).

### Mode for Invention

Hereinafter, the formulating methods and kinds of excipients will be described, but the present invention is not limited to them. The representative preparation examples were described as follows.

### 1. Preparation of Powder

| | | |
|---|---|---|
| CINTM1 | 20 | mg |
| Lactose | 100 | mg |
| Talc | 10 | mg |

Powder preparation was prepared by mixing above components and filling sealed package.

### 2. Preparation of Tablet

| | | |
|---|---|---|
| CINTM1 | 10 | mg |
| Corn Starch | 100 | mg |
| Lactose | 100 | mg |
| Magnesium stearate | 2 | mg |

Tablet preparation was prepared by mixing above components and entabletting.

### 3. Preparation of Capsule

| | | |
|---|---|---|
| CINTM1 | 10 | mg |
| Crystalline cellulose | 3 | mg |
| Lactose | 14.8 | mg |
| Magnesium stearate | 0.2 | mg |

Capsule preparation was prepared by mixing above components and filling gelatin capsule by conventional gelatin preparation method.

### 4. Preparation of Injection

| | | |
|---|---|---|
| CINTM1 | 10 | mg |
| Mannitol | 180 | mg |
| Sterilized distilled water for injection | 2974 | mg |
| Na₂HPO₄—12H₂0 | 26 | mg |

Injection preparation was prepared by dissolving active component, and then filling all the components in 2 ml per ample by conventional injection preparation method.

### 5. Preparation of Liquid

| | | |
|---|---|---|
| CINTM1 | 20 | mg |
| Isomerized glucose | 10 | g |
| Mannitol | 5 | g |

| | |
|---|---|
| distilled water | optimum amount |

Liquid preparation was prepared by dissolving active component in distilled water, adding lemon flavor, mixing together with distilled water and then filling all the components in 100 ml brown bottle and sterilizing by conventional liquid preparation method.

### 6. Preparation of Health Food

| CINTM1 | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in many ways. Such variations are not to be regarded as a departure from the spirit and scope of the present invention.

### 7. Preparation of Health Beverage

| | | |
|---|---|---|
| CINTM1 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 2 L ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the combined herb composition shows potent treating effect on human infertility, which is confirmed by various experiments, for example, In vitro sperm quality promoting effect evaluation as well as in vivo varicocele animal model evaluation method for investigating effects of the herbal extract on changes in sperm count and mobility in an in vivo varicocele animal model, it was verified that the inventive combined extract showed more potent increasing effect on the sperm count and sperm motility. Therefore, the herbal extract of the present invention can be usefully used in a pharmaceutical composition, health functional food, and health supplement food for preventing and treating male infertility.

1. A pharmaceutical composition comprising a combination of extracts from Morinda Officinalis root, Cuscuta seed, and Allium cepa bulb as an active ingredient for treating male infertility.

2. The pharmaceutical composition of claim 1, wherein the combination of extracts from Morinda Officinalis root, Cuscuta seed, and Allium cepa bulb has a range of mixed ratios based on the dried weight of each herb of 0.01-100:0.01-1:1-100 (w/w).

3. The pharmaceutical composition of claim 1, wherein the male infertility is selected from the group consisting of an adverse response of an anti-cancer agent, azoospermia, oligospermia, asthenospermia, and teratospermia.

4. A health functional food comprising a combination of extracts from Morinda Officinalis root, Cuscuta seed, and Allium cepa bulb as an active ingredient for the improvement of male infertility.

5. The health functional food of claim 4, wherein the health food is selected from the group consisting of powder, granule, tablet, capsule, pill, suspension, emulsion, syrup, tea bag, leached tea, or beverage type.

6. (canceled)

7. A method of treatment of male infertility comprising administering the pharmaceutical composition of claim 1 to a subject in need of treatment of male infertility.

8. (canceled)

9. The method of claim 7, further comprising the step of:
obtaining extracts from Morinda Officinalis root, Cuscuta seed, and Allium cepa bulb;
mixing the extracts together to obtain the combination of extracts from Morinda Officinalis root, Cuscuta seed, and Allium cepa bulb; and
administering the combination of extracts to the subject in need of treatment of male infertility.

10. The method of claim 8, wherein the combination of extracts from Morinda Officinalis root, Cuscuta seed, and Allium cepa bulb has a range of mixed ratios based on the dried weight of each herb of 0.01-100:0.01-1:1-100 (w/w).

11. The method of claim 7, wherein the male infertility is selected from the group consisting of an adverse response of an anti-cancer agent, azoospermia, oligospermia, asthenospermia, and teratospermia.

12. A method of manufacturing a medicament for the treatment of male infertility in a human or mammal comprising the steps of
extracting a composition from Morinda Officinalis root,
extracting a composition from Cuscuta seed, and
extracting a composition from Allium cepa bulb; and
mixing the compositions together at mixed ratios ranging from 0.01-100:0.01-1:1-100, based on the dried weight of each composition (w/w).
